(19) United States
(12) Patent Application Publication
(10) Pub. No.: US 2012/0328529 A1
(43) Pub. Date: Dec. 27, 2012

(54) MICROFLUIDIC-BASED GENERATION OF FUNCTIONALIZED MICROBUBBLES FOR ULTRASOUND IMAGING AND THERAPY

(75) Inventors: Daeyeon Lee, Wynnewood, PA (US); Chandra M. Sehgal, Wayne, PA (US); Myung Han Lee, Seoul (KR)

(73) Assignee: THE TRUSTEES OF THE UNIVERSITY OF PENNSYLVANIA, Philadelphia, PA (US)

(21) Appl. No.: 13/388,858

(22) PCT Filed: Aug. 5, 2010
(86) PCT No.: PCT/US10/44555

§ 371 Date: Sep. 7, 2012

Related U.S. Application Data
(60) Provisional application No. 61/231,558, filed on Aug. 5, 2009.
(60) Provisional application No. 61/367,120, filed on Jul. 23, 2010.

(51) Int. Cl. A61K 49/22; A61K 9/14; A61K 9/00

(52) U.S. Cl. 424/9.5; 424/400; 264/4

(57) ABSTRACT

A method for generating stable encapsulated bubbles and dried encapsulated bubbles comprises introducing an inner stream of a gas into a liquid-filled chamber from an exit orifice of a capillary tube; introducing a middle stream of a water immiscible liquid into the exit orifice; and introducing an outer stream of an aqueous liquid to the exit orifice, to form compound bubbles. The encapsulated bubbles are formed after converting the middle phase of compound bubbles into a shell. The stable encapsulated bubbles or the stable dried encapsulated bubbles can be used in drug delivery and for enhancing ultrasound imaging.

### RELATED APPLICATIONS

This application claims priority under 35 U.S.C. §119(e) to U.S. Provisional Application No. 61/231,558, filed on Aug. 5, 2009, and U.S. Provisional Application No. 61/367,120, filed on Jul. 23, 2010, the contents of both of which are incorporated by reference herein, in their entirety and for all purposes.

### FIELD OF THE INVENTION

The invention relates generally to generation of stable encapsulated bubbles and the use thereof. More particularly, the invention relates to methods for generating stable encapsulated bubbles and dried encapsulated bubbles generated therefrom.

### BACKGROUND OF THE INVENTION

A bubble is a globular body of gas suspended or dispersed in a liquid. Monodisperse, stable bubbles can be used in biomedical applications such as contrast-enhanced ultrasonography and drug delivery. Stable and monodisperse bubbles also have potential applications in the fabrication of functional lightweight materials with hierarchical structure, the encapsulation of flavors and fragrances for food and cosmetics additives. Bubbles which remain stable when dry are particularly advantageous in that the cost for shipping such bubbles and the ease of handling them is significantly reduced.

Conventional methods for preparing bubbles involve the production of a dispersion of gas in a solution containing amphiphilic molecules such as phospholipids and surfactants by sonication, mechanical agitation or high shear mixing. Bubbles generated using these methods tend to be very polydisperse in size. A recently developed electric bubble generation method provides a better control of size distribution, but nevertheless generates bubbles with a polydispersity index (δ) of 30-40% (δ(%)=σ/D×100 where σ and D are the standard deviation and average diameter of bubbles, respectively). In addition to their polydispersity, gas bubbles stabilized by amphiphilic molecules undergo dissolution and coarsening via Ostwald ripening due to the effects of Laplace pressure across the air-water interface, making it very difficult to retain size uniformity for an extended period of time.

Recently, microfluidic approaches have been utilized for generating stable and monodisperse bubbles with controlled dimensions. The polydispersity (δ) of these bubbles is typically on the order of 5%. In these approaches, monodisperse bubbles generated in a microfluidic device are stabilized by the adsorption of amphiphilic molecules or partially hydrophobic particles from an aqueous phase to the air-water interface. In particular, the stability of bubbles has been drastically improved by the formation of close-packed layers of colloidal particles at the air-water interface. Monodisperse bubbles with a hydrogel shell suspended in oil have also been generated using a microfluidic technique. A hydrogel shell was generated by photopolymerizing water soluble monomers to encapsulate gas bubbles. These approaches, however, are limited to the use of amphiphilic or water dispersible materials for stabilization of the air-water interface. This limitation restricts the formation of composite shells that are composed of mixtures of hydrophobic molecules and nanoparticles with unique properties. The incorporation of non-water soluble materials such as drugs and nanomaterials would significantly enhance the versatility of composite bubbles in various applications.

There exists a need for methods of generating stable gas bubbles with high uniformity in size and properties, which can be stored for an extended period of time without significant changes in size and other properties, and preferably can be stored in a dried state and then resuspended without a loss of a significant number of the bubbles.

### SUMMARY OF THE INVENTION

The invention provides a method for forming stable encapsulated bubbles. In general, the method comprises (a) introducing an inner stream of a gas into a liquid-filled chamber from a circular exit orifice of a capillary tube; (b) introducing, after step (a), a middle stream of a water immiscible liquid to the exit orifice within the chamber, wherein the water immiscible liquid comprises a shell forming material; (c) introducing, after step (a), an outer stream of an aqueous liquid to the exit orifice within the chamber, wherein the gas, the water immiscible liquid, and the aqueous liquid interact to form compound bubbles having an inner phase of the gas, a middle phase of the water immiscible liquid, and an outer phase of the aqueous liquid; and (d) converting the middle phase of the compound bubbles of step (c) into a shell, wherein the shell encapsulates the gas and is surrounded by the aqueous liquid, to form stable encapsulated bubbles. The middle stream may be introduced in step (b) before the outer stream is introduced in step (c). The method may further comprise storing the encapsulated bubbles for an extended period of time, for example, one year. The diameter of the circular exit orifice is preferably in the range of 2-5 μm.

In some embodiments, the method further comprises modifying at least one of: (a) the ratio of the shell thickness to the bubble radius; (b) the stiffness of the bubble shell; and (c) the identity of the gas; to improve the stability of the encapsulated bubbles. In one embodiment, where the modifying step consists of modifying the ratio of the shell thickness to the bubble radius, modifying the ratio of the shell thickness to the bubble radius consists of adjusting the relative volumetric flow rates of at least one of the inner stream, the middle stream, and the outer stream.

In other embodiments, the water immiscible liquid further comprises a solvent and the conversion step comprises (a) collecting the compound bubbles substantially in a monolayer; and (b) removing the solvent from the middle phase of the bubbles in the monolayer to convert the middle phase into the shell.

The encapsulated bubbles may have high uniformity in size or other properties (e.g., polydispersity, shape, percent fracture, echogenicity and mechanical properties). For example, the encapsulated bubbles may have a polydispersity of less than 2%, preferably less than 1%, more preferably less than 0.5%, and most preferably less than 0.3%.

The gas may be any gas or any combination of two or more gases. Preferably, the gas is relatively insoluble in the aqueous liquid of the outer stream. The gas is also preferably similar to air to avoid excessive diffusion of the gas out of the bubble.

The water immiscible liquid may further comprise an active pharmaceutical ingredient. The bubbles of the present invention provide a carrier for delivering an active pharmaceutical ingredient which is hydrophobic or less than highly soluble in water. The active pharmaceutical ingredient may be present in a pharmaceutically effective amount in the water immiscible liquid. The encapsulated bubbles may be used to deliver the active pharmaceutical ingredient to a subject.

The water immiscible liquid may also further comprise nanoparticles. The nanoparticles may be magnetic, and responsive to a magnetic field. The encapsulated bubbles comprising nanoparticles may be used as an ultrasound agent to enhance ultrasound imaging in a subject. The encapsulated bubbles comprising a magnetic, fluorescent or radiopaque material (e.g., nanoparticles) may be used as a dual agent for ultrasound in combination with magnetic resonance, X-ray, optical or photoacoustic.

The present invention also provides a method of forming stable dried encapsulated bubbles, comprising (a) introducing an inner stream of a gas into a liquid-filled chamber from a circular exit orifice of a capillary tube; (b) introducing, after step (a), a middle stream of a water immiscible liquid to the exit orifice within the chamber, wherein the water immiscible liquid comprises a shell forming material; (c) introducing, after step (a), an outer stream of an aqueous liquid to the exit orifice within the chamber, wherein the gas, the water immiscible liquid, and the aqueous liquid interact to form compound bubbles having an inner phase of the gas, a middle phase of the water immiscible liquid, and an outer phase of the aqueous liquid; (d) converting the middle phase of the compound bubbles of step (c) into a shell, wherein the shell encapsulates the gas and is surrounded by the aqueous liquid, to form stable encapsulated bubbles; and (e) removing the aqueous liquid from the stable encapsulated bubbles of step (d) to form stable dried encapsulated bubbles.

The method may further comprise re-dispersing the dried encapsulated bubbles in an aqueous solution to form stable re-dispersed encapsulated bubbles. The dried bubbles may be stored before redispersion. Upon re-dispersion, most, for example, at least 80%, of the dried encapsulated bubbles form stable re-dispersed encapsulated bubbles.

Also provided by the present invention are compositions comprising the stable encapsulated bubbles, the stable dried encapsulated bubbles, and the stable re-dispersed encapsulated bubbles obtained by the methods of the present invention.

The present invention also provides a method of treating a subject comprising administering to the subject a composition comprising the encapsulated bubbles obtained by the method of the present invention, wherein the water immiscible liquid comprises an active pharmaceutical ingredient and the active pharmaceutical ingredient is present in a therapeutically effective amount. The composition may be administered by a route selected from the group consisting of oral, subcutaneous, intravenous, intranasal, transdermal, intraperitoneal, intramuscular, intrapulmonary, vaginal, rectal, and intraocular routes. The method may further comprise delivering the encapsulated bubbles to a target location in the subject and releasing the active pharmaceutical ingredient from the encapsulated bubbles at the target location.

The present invention provides a method of enhancing ultrasound imaging in a subject comprising administering to the subject a composition comprising the encapsulated bubbles obtained by the method of the present invention, wherein the water immiscible liquid further comprises nanoparticles.

### BRIEF DESCRIPTION OF THE DRAWINGS

FIGS. 1(a) and 1(b) show a schematic diagram and an optical microscopy image of air-in-oil-in-water (A/O/W) compound bubbles generated in a capillary microfluidic device, respectively. FIG. 1(c) shows the dependence of the encapsulated bubble size (D_{bubble}) (open square) and oil thickness (Lₒᵢₗ) (closed square) on the flow rates of middle (Qₘ) and outer phases (Qₒ) and the pressure of inner phase (Pᵢ). D_{bubble} and Lₒᵢₗ were determined by the oil flow rate (Qₘ), compound-bubble generation frequency (f_{bubble}), and compound-bubble size (Dₐᵥₑ), which were given by D_{bubble}=(Dₐᵥₑ³−6Qₘ/f_{bubble}π)^{1/3} and Lₒᵢₗ=(Dₐᵥₑ−D_{bubble})/2, respectively.

FIG. 2(a) shows a schematic diagram of the formation of bubbles with the rigid shell of close-packed nanoparticles from microfluidic-generated A/O/W compound bubbles. FIG. 2(b) shows long-term stability of bubbles generated at Qₒ=120,000 μl/hr, Qₘ=1,000 μl/hr, and Pᵢ=82.7 kPa (left), and an optical microscopy image of bubbles 1020 hours after preparation (right). The inset is a histogram showing the size distribution of bubbles.

FIG. 3 shows SEM images of hollow shells obtained after bubbles generated at Qₒ=120,000 μl/hr, Qₘ=1,000 μl/hr, and Pᵢ=82.7 kPa were completely air-dried at room temperature.

FIGS. 4(a)-(c) show generation of bubbles with multi-composite shells. FIG. 4(a) shows optical images of magnetic bubbles moving parallel to a magnetic field gradient (left), and x- and y-trajectories as a function of time for the magnetic bubble motion induced by the magnetic field gradient (right). FIG. 4(b) shows a fluorescent image of bubbles with the silica shell containing the fluorescent dye, Nile Red. FIG. 4(c) shows an SEM image of air-dried bubbles with a palmitic acid/silica nanoparticle composite shell.

FIGS. 5(a)-(d) shows microfluidic fabrication of PLGA-shelled bubbles. FIG. 5(a) shows a schematic diagram for the formation of polymer-shelled bubbles from A/O/W compound bubbles. FIG. 5(b) shows an optical microscopy image of A/O/W compound bubbles generated in a microfluidic device. FIGS. 5(c) and 5(d) show optical microscopy images of PLGA-shelled bubbles 30 minutes after preparation. Bubbles have (c) R=40.9 μm and h=104 nm and (d) R=19.3 μm and h=93 nm. R and h were determined by the mass balance using oil flow rate (Qₘ), compound-bubble generation frequency (f_{cb}), volume fraction of polymer in oil (φₚ), and compound-bubble size (D_{cb}). Insets show the SEM images of polymer shells after being completely air-dried at room temperature. Scale bars are 100 μm.

FIG. 6 shows a state diagram for bubble stability as functions of the shell thickness (h) and bubble radius (R), determined at 30 minutes after preparation. The symbols depend on the percentage of deformed bubbles: (▪) 0-10%; (□) 10-25%; (▴) 25-50%; (□) 50-75%; (▾) 75-100%. The dashed line goes through the origin of the h versus R graph and has a slope of (h/R)_{c}. Optical microscopy images correspond to the several data points (a, b and c) on the diagram.

FIG. 7 shows the percentage of deformed bubbles as a function of h/R at 30 minutes after preparation and (inset) 2 days after preparation.

FIGS. 8(a) and 8(b) shows the percentage of deformed bubbles with the PLGA-, PS-, and PMMA-shells as a function of (a) h/R and (b) E_{f}(h/R)².

FIGS. 9(A)-(C) show size distribution of dried encapsulated bubbles in Sample 1 (A) before drying and (B) after re-dispersion as well as (C) before and after re-dispersion.

FIGS. 10(A)-(C) show size distribution of dried encapsulated bubbles in Sample 2 (A) before drying and (B) after re-dispersion as well as (C) before and after re-dispersion.

FIGS. 11(A)-(D) show images of dried encapsulated bubbles ((A) and (C)) and remaining bubbles ((B) and (D)) after re-dispersion for Sample 1 ((A) and (B)) and Sample 2 ((C) and (D)).

FIGS. 12(A) and 12(B) show images of re-dispersed sample 1 (A) and re-dispersed Sample 2 (B).

### DETAILED DESCRIPTION OF THE INVENTION

The present invention generally relates to methods of forming stable encapsulated bubbles and stable dried encapsulated bubbles, and to methods of using these bubbles. The encapsulated bubbles may be formed to have three phases by combining an inner phase of a gas, a middle phase of a water immiscible liquid comprising a shell forming material, and an outer phase of an aqueous liquid, followed by converting the middle phase into a shell. The resulting encapsulated bubbles have a shell encapsulating the gas and surrounded by the aqueous liquid. The encapsulated bubbles may be used to carry or deliver a functional material in the shell. The encapsulated bubbles may be dried and stored without any significant change in size or other properties (e.g., polydispersity, shape, percent fracture, echogenicity and mechanical properties) for an extended period of time.

In some embodiments, the stable encapsulated bubbles are formed by (a) introducing an inner stream of a gas into a liquid-filled chamber from a circular exit orifice of a capillary tube; (b) introducing, after step (a), a middle stream of a water immiscible liquid to the exit orifice within the chamber, wherein the water immiscible liquid comprises a shell forming material; (c) introducing, after step (a), an outer stream of an aqueous liquid to the exit orifice within the chamber, wherein the gas, the water immiscible liquid, and the aqueous liquid interact to form compound bubbles having an inner phase of the gas, a middle phase of the water immiscible liquid, and an outer phase of the aqueous liquid; and (d) converting the middle phase of the compound bubbles of step (c) into a shell, wherein the shell encapsulates the gas and is surrounded by the aqueous liquid, to form stable encapsulated bubbles. The middle stream may be introduced in step (b) before the outer stream is introduced in step (c).

As used herein, the term “stable encapsulated bubbles” means that the encapsulated bubbles do not exhibit any substantial change in size, shape, or percent fractured over an extended period of time. By not exhibiting any “substantial change,” the bubbles exhibit a change in the relevant property identified by numerical values (e.g., size and polydispersity) of less than about 50%, more preferably less than about 30%, more preferably less than about 10%, even more preferably less than about 5%, still more preferably less than about 2% and most preferably less than about 1%. The period of time during which the bubbles remain stable will depend on application of the bubbles, but preferably is at least 1 hour, one day, or two days, more preferably at least one week, even more preferably at least one month, still more preferably at least six months and most preferably at least one year. Preferably, the encapsulated bubbles do not undergo any substantial change over an extended period of time in other properties, such as echogenicity and other mechanical and dynamic properties. One example of a dynamic property relevant to the use of the bubbles in ultrasound is the relationship between the volumetric change of the bubbles as a function of frequency applied to the bubbles via ultrasound.

The inner phase may be any gas (e.g., nitrogen, carbon dioxide, or helium) or a combination of two or more gases (e.g., compressed air) and will depend on the particular application of the bubbles as is known in the art. The stability of the encapsulated bubbles may be improved by modifying the identity of the gas. As described in the Examples below, it is believed that a pressure drop across the bubbles leads to bubble deformation. Therefore, to improve stability, one should select a gas that has a low solubility in the aqueous liquid. Even more preferably, to improve stability, one should select a gas that is similar compositionally to air to minimize diffusion. Some gases that work well are air, nitrogen, or a very hydrophobic gas, while others that do not work very well include carbon dioxide, helium, ammonia, or a very hydrophilic gas.

The middle phase may be amphiphilic and preferably is hydrophobic. Where the middle phase comprises an oil, the compound bubbles may be characterized as air-in-oil-in-water (A/O/W) compound bubbles. The water immiscible liquid comprises a shell forming material. The shell forming material is one that, upon conversion, coalesces and forms a stable shell of the bubble, which may be disrupted by a suitable external stimulus (e.g., ultrasound, heat, pressure, magnetic stimulus) or under certain physiological conditions. Shell forming materials can be dispersed in the water immiscible liquid. Typical shell forming materials are nanoparticles, silica particles (preferably silica nanoparticles), polymers, clay particles, monomers, crystallizable liquids, and phase changing liquids. A phase changing liquid can change phases, for example, from liquid to solid, upon a temperature change or curing, for example by radiation. For example, a wax or gel may be used as the shell forming material in making bubbles in the chamber, which is maintained at a temperature at which the wax or gel is liquid. Upon removal of the bubbles from the chamber, the wax or gel solidifies such that the middle phase is converted into a shell encapsulating the gas.

The water immiscible liquid may further comprise a solvent in which a shell forming material is dispersed or dissolved. The bubbles may be collected substantially in a monolayer at the surface of an aqueous liquid (e.g., water). The term “substantially” as used in this manner means that at least about 60%, more preferably 70%, more preferably 80%, still more preferably 90%, even more preferably 95% and most preferably 99% are collected in a reservoir such that the bubbles are in a single layer, not overlapping with each other. This can be achieved by using a collection reservoir that is sized to achieve this effect of having the bubbles in a single layer. The solvent is preferably organic and more volatile than the aqueous liquid, and may be removed from the middle phase of the bubbles by evaporation or washing away such that the middle phase is converted into a shell encapsulating the gas to form stable encapsulated bubbles. Examples of the solvent include toluene, chloroform and dichloromethane, and fluorocarbons generally.

The stability of the encapsulated bubbles may be improved by modifying the stiffness of the shell, for example, by selecting a shell forming material that has intrinsically low Young's modulus and/or that does not swell too much in water. As another alternative to modifying the stiffness of the shell, a plasticizer, such as citrates, could be added to the water immiscible liquid. Of course, the desired stiffness (and stability) is dependent upon the ultimate use of the bubbles, so one might wish to reduce the stiffness of the bubbles by, for example, selecting a shell forming material having a relatively higher Young's modulus and/or one that does swell significantly in water.

The water immiscible liquid may comprise, optionally, a functional material. Examples of the functional materials include an active pharmaceutical ingredient, a diagnostic agent, nanoparticles, polymers, lipids, metal, ceramic, magnetic, radiopaque or fluorescent materials, and mixture thereof. Preferably, the functional material is hydrophobic or insoluble in water. The functional material may be pre-mixed in the water immiscible liquid prior to being introduced into the chamber, or added to the immiscible liquid while being introduced into the chamber. In fact, a single material may serve both as the shell-forming material and the functional material. For example, nanoparticles of any known material (e.g., silica, carbon, oxide, metal, or semiconductor) may be a shell forming material and/or a functional material. Different nanoparticles may be mixed (e.g., fluorescent+silica, magnetic+silica, and porous+solid silica) to form desirable stable encapsulated bubbles.

The outer phase is designed to prevent or minimize contact among bubbles having the shell encapsulating the gas, especially before the bubbles cure or harden. It can be water alone but preferably includes an additive that enhances the stability of the bubbles. One such additive is poly(vinyl alcohol), which was found to prevent the coalescence of the generated encapsulated bubbles with one another. Other stability-enhancing additives include surfactants, proteins, and ligands. The amount of the stability enhancer is that amount which provides the stable bubbles. Typically, PVA ranges from about 0.1 to 10 wt %, preferably from about 1 to 5 wt %.

To generate thermodynamically stable core-shell structures from three fluids, the spreading coefficient, defined as Sᵢ=γⱼₖ−γᵢⱼ−γᵢₖ (i≠j≠k=op, ip, mp) (where γⱼₖ, γᵢⱼ, and γᵢₖ are the surface and interfacial tensions between j-k, i-j, and i-k fluids), of three phases must satisfy the following relationship: Sₒₚ<0, Sᵢₚ<0, and Sₘₚ>0 (the subscript op, ip, and mp indicate the outer, inner, and middle phases, respectively). In our system, the spreading coefficients, determined using pendant drop tensiometry, were, for example, Sₒₚ=−27.1<0 mN/m, Sᵢₚ=−66.4<0 mN/m, and Sₘₚ=18.7>0 mN/m, satisfying conditions for the engulfment of one dispersed phase in the second phase. More preferably, the three phases satisfy the following relationship: Sₒₚ<−10, Sᵢₚ<−30, and Sₘₚ>10.

The encapsulated bubbles may be formed according to the methods of the present invention in any chamber comprising a capillary tube from a circular exit orifice of which the gas is introduced into the chamber. The diameter of the circular exit orifice may be in the range of 0.1-100 μm, preferably in the range of about 2-5 μm.

The stability of the encapsulated bubbles may be improved by modifying the ratio of the shell thickness (h) to the bubble radius (R). This ratio may be modified by adjusting the relative volumetric flow rates of at least one of the inner stream, the middle stream and the outer stream. The ratio may also be modified by selecting different shell forming materials, adjusting the dimension of the exit orifice of the capillary tube, and the pressure of the gas. For example, with all else being constant, as the volumetric flow rate of the middle stream is increased, an increased shell thickness will result, in general. For a selected combination of the gas, the water immiscible liquid and the aqueous liquid, a critical ratio, (h/R)_{c}, for the onset of bubble shell deformation can be determined empirically, for example, as illustrated below in Example 5.

The encapsulated bubbles have a radius in the range of about 0.1-100 μm, preferably in the range of about 0.5-5 μm. The encapsulated bubbles may be made smaller by several ways. For example, a highly soluble gas may be selected as the inner phase to reduce the bubbles into a desirable size range (e.g., 1-5 μm) by diffusing the gas into the outer phase through the middle phase or by exposing the compound bubbles to a low pressure environment before the middle phase is converted into a shell. The shell thickness is in the range of about 10 nm to about 10 μm, preferably about 10 nm to about 1 μm. The encapsulated bubbles have a polydispersity of less than about 5%, 3% or 1%, preferably less than about 3%.

In other embodiments, stable dried encapsulated bubbles are formed by (a) introducing an inner stream of a gas into a liquid-filled chamber from a circular exit orifice of a capillary tube; (b) introducing, after step (a), a middle stream of a water immiscible liquid to the exit orifice within the chamber, wherein the water immiscible liquid comprises a shell forming material; (c) introducing, after step (a), an outer stream of an aqueous liquid to the exit orifice within the chamber, wherein the gas, the water immiscible liquid, and the aqueous liquid interact to form compound bubbles having an inner phase of the gas, a middle phase of the water immiscible liquid, and an outer phase of the aqueous liquid; (d) converting the middle phase of the compound bubbles of step (c) into a shell, wherein the shell encapsulates the gas and is surrounded by the aqueous liquid, to form stable encapsulated bubbles; and (e) removing the aqueous liquid from the stable encapsulated bubbles of step (d) to form stable dried encapsulated bubbles. This method may further comprise re-dispersing (or resuspending) the dried encapsulated bubbles in an aqueous solution to form re-dispersed encapsulated bubbles. The aqueous solution may be the same as the aqueous liquid used to form the dried encapsulated bubbles. The dried encapsulated bubbles may be stored for an extended period of time (e.g., at least one hour, one day, one week, four weeks, one month, six months or one year) before re-dispersion.

In some embodiments, upon re-dispersion, at least about 60%, more preferably about 70%, more preferably about 80%, even more preferably about 90% or most preferably about 95%, of the dried bubbles form encapsulated bubbles. As used herein, the term “stable dried bubbles” means that the dried bubbles do not exhibit any substantial change in size, shape, or percent fractured over an extended period of time when compared with the encapsulated bubbles from which the dried encapsulated bubbles are made or before drying. By not exhibiting any “substantial change,” the bubbles exhibit a change in the relevant property identified by numerical values (e.g., size and polydispersity) of less than about 50%, more preferably less than about 30%, more preferably less than about 10%, even more preferably less than about 5%, still more preferably less than about 2% and most preferably less than about 1%. The period of time during which the bubbles remain stable will depend on application of the bubbles, but preferably is at least 1 hour, one day, or two days, more preferably at least one week, even more preferably at least one month, still more preferably at least six months and most preferably at least one year. Preferably, the dried bubbles do not undergo any substantial change over an extended period of time in other properties, such as echogenicity and other mechanical and dynamic properties. One example of a dynamic property relevant to the use of the bubbles in ultrasound is the relationship between the volumetric change of the bubbles as a function of frequency applied to the bubbles via ultrasound.

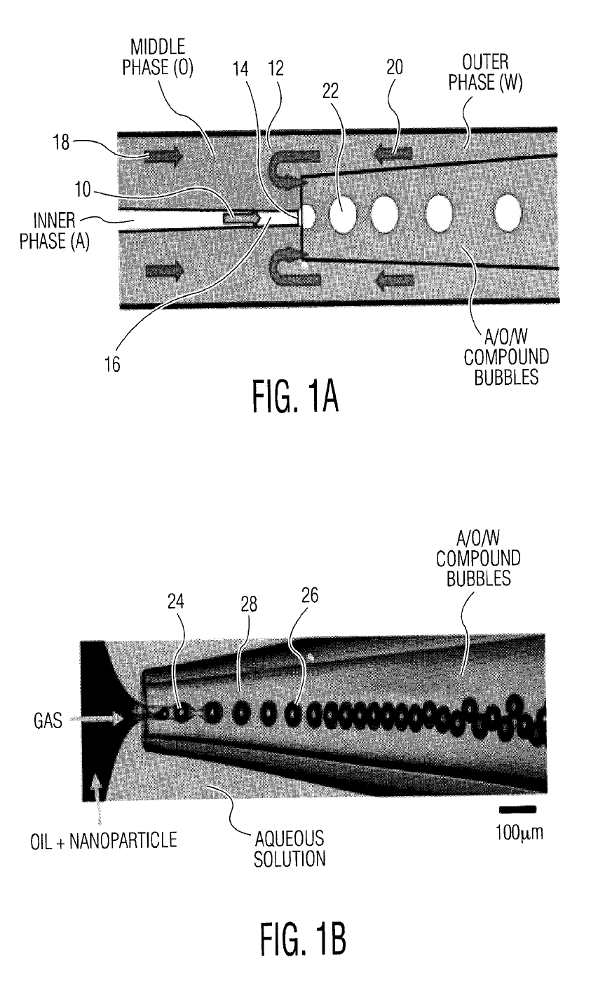

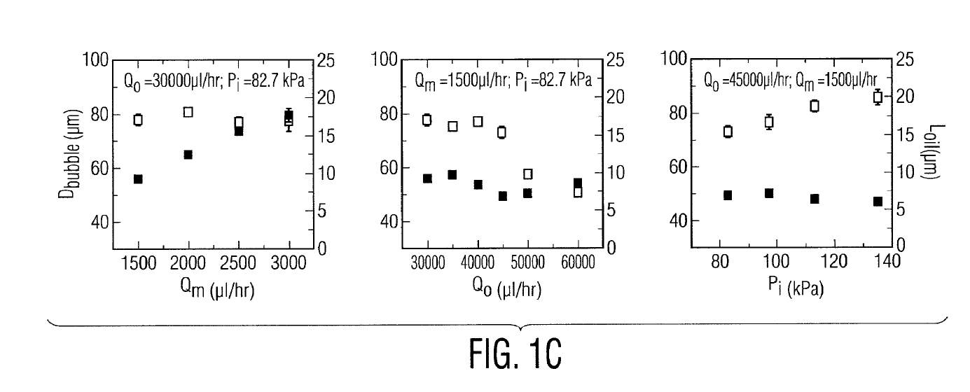

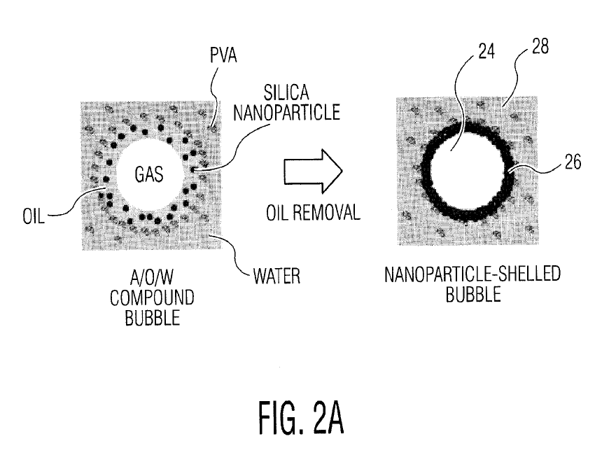

Any suitable conventional apparatus can be used to make the bubbles of the present invention, such as that described in U.S. Patent Application Publication No. 2009/0131543 to Weitz et al., entitled METHOD AND APPARATUS FOR FORMING MULTIPLE EMULSIONS, incorporated herein by reference. The method for making the stable encapsulated bubbles can be described in connection with the schematic drawing shown in FIG. 1(a). As mentioned above, first an inner stream 10 of gas is introduced into chamber 12 from circular exit orifice 14 of capillary tube 16. Then, a middle stream 18 of the water immiscible liquid is introduced to circular exit orifice 14 within chamber 12, and an outer stream 20 of an aqueous liquid is introduced to circular exit orifice 14 within chamber 12, as shown in FIGS. 1(a) and 1(b), in such a way that the gas, the water immiscible liquid, and the aqueous liquid interact to form compound bubbles 22 having an inner phase 24 of the gas, a middle phase 26 of the water immiscible liquid, and an outer phase 28 of the aqueous liquid, as shown in FIG. 2(a). The middle phase 26 is subsequently converted into a shell encapsulating the gas and surrounded by the outer phase 28. The middle stream 18 may be introduced before or after the outer stream 20 is introduced. It has been found particularly preferable to introduce inner stream 10 before middle stream 18 or outer stream 20 is introduced upon starting up. Before introduction of any of the three phases, the chamber is filled with liquid (e.g., water). It has also been found preferable to avoid any of the water immiscible liquid or the aqueous liquid to flow backwards into capillary tube 16. This can be accomplished by applying a small amount of pressure to the gas (e.g., 0.5 psi) prior to connecting the sources of the middle phase and the outer phase to chamber 12. Where a collection tube is used to collect the compound bubbles, the gas is flowed out from the exit orifice before the middle phase reaches the entrance of the collection tube to avoid clogging the exit orifice

The present invention provides compositions comprising the encapsulated bubbles, the dried encapsulated bubbles or the re-dispersed (or resuspended) encapsulated bubbles obtained by the methods of the present invention. The bubbles may comprise a functional material. These encapsulated bubbles have broad applications, especially in the biomedical field (e.g., drug delivery, ultrasound, magnetic resonance, and pressure sensing). For example, the encapsulated bubbles comprising a magnetic, radiopaque or fluorescent material may be used for dual imaging (e.g., ultrasound in combination with magnetic resonance, X-ray, optical or photoacoustic). Also, the encapsulated bubbles prepared by the methods of the present invention have high uniformity in size, and may be introduced into blood stream as an in vivo pressure sensor to detect resonance change, which reflects bubble size change due to blood pressure change during a cardiac cycle.

The present invention also provides a method for treating a subject comprising administering to the subject a composition comprising encapsulated bubbles obtained by the method of the present invention, wherein the shell comprises an active pharmaceutical ingredient in a therapeutically effective amount. The subject may be an animal, preferably a human, more preferably, a patient in need of a treatment for which the active pharmaceutical ingredient is suitable. The composition is administered by a route selected from the group consisting of oral, subcutaneous, intravenous, intranasal, transdermal, intraperitoneal, intramuscular, intrapulmonary, vaginal, rectal, and intraocular routes. The method may further comprise delivering the encapsulated bubbles to a target location in the subject; and releasing the active pharmaceutical ingredient from the encapsulated bubbles at the target location. The delivery of the encapsulated bubbles to the target location may be guided by ultrasound imaging. The release of the active pharmaceutical ingredient from the bubbles may be triggered by a suitable external stimulus (e.g., ultrasound, heat, pressure, magnetic stimulus). The release of the active pharmaceutical ingredient may also be controlled in a timely manner by, for example, providing a bubble shell having desirable pharmacokinetic properties. Alternatively, an active pharmaceutical ingredient which is less than highly soluble in water can be mixed with the water immiscible liquid to form encapsulated bubbles which may be formulated in an oral dosage form. The stability of the bubbles can be tuned to release the dosage form after an particular time after ingestion.

The present invention further provides a method of enhancing ultrasound imaging in a subject, comprising administering to the subject a composition comprising encapsulated bubbles obtained by the method of the present invention, wherein the shell further comprises nanoparticles. The subject may be an animal, preferably a human, more preferably a patient in need of ultrasound imaging. The method may further comprising performing other imaging (e.g., MRI, X-ray, optical and photoacoustic) on the subject, wherein the nanoparticles are magnetic, radiopaque or fluorescent.

The term “about” as used herein when referring to a measurable value such as an amount, a percentage, and the like, is meant to encompass variations of ±20% or ±10%, preferably ±5%, more preferably ±1% from the special value, as such variations are appropriate to perform the disclosed methods.

The following examples are provided to describe exemplary aspects of the invention in greater detail. They are intended to illustrate, not to limit, the invention.

### Example 1

### Generation of Nanoparticle-Shelled Bubbles

The glass-capillary microfluidic device with a hydrodynamic flow-focusing and co-flowing geometry was fabricated. Two circular capillary tubes with inner and outer diameters of 0.58 mm and 1.0 mm (World Precision Instrument Inc.) were tapered for the injection of a gas phase and the collection of bubbles, using a micropipette puller (P-1000, Sutter Instrument Inc.) and a microforge (Narishige MF-830). The diameter of the inner orifice ranged from 2 to 5 μm and that of the collection tube ranged from 100 to 150 μm. The two capillaries were inserted into a square capillary with an inner dimension of 1.0 mm.

For the generation of air-in-oil-in-water (A/O/W) compound bubbles with a gas-core and oil-shell suspended in water, two liquids and one gas were introduced into a microfluidic device. Nitrogen (N₂) as the inner phase was delivered into the device via flexible Tygon tubing with a pressure regulator (ControlAir Inc.). A 2 wt % polyvinyl alcohol) aqueous solution (PVA, 87-89% hydrolyzed, average M_{w}=13,000-23,000, Aldrich) as the outer phase and a toluene solution containing hydrophobic silica nanoparticles (average diameter=˜15 nm, Nisan Chemical Inc. Tol-ST) as the middle phase were loaded in two syringes (SGE or Hamilton Gastight) and introduced into the device using syringe pumps (PHD, Harvard Apparatus). For the fabrication of bubbles with magnetic, fluorescent, and free fatty acid-containing shells, the middle oil phases were prepared by dissolving 14 μl of toluene-based ferrofluid (Ferrotec Corp.), 20 μl of toluene-based Nile Red solution (1 mg/ml), and 500 μl of toluene solution containing palmitic acid (100 mg/ml) in 5 ml of toluene solution containing silica particles (volume fraction=0.12), respectively.

In this work, monodisperse nanoparticle-shelled bubbles were created using an air-in-oil-in-water (A/O/W) compound bubble as a template. These A/O/W compound bubbles were generated using a glass-capillary microfluidic device combining both a hydrodynamic flow-focusing and co-flowing geometry, as shown in FIG. 1(a). The inner phase (A) was nitrogen, and the outer phase (W) was a 2 wt % poly(vinyl alcohol) (PVA) aqueous solution. The middle phase (O), which was immiscible with both the inner and outer phases, was a toluene solution containing hydrophobic silica nanoparticles. The outer phase hydrodynamically focused the middle and inner fluid streams, breaking them into monodisperse A/O/W compound bubbles with a core-shell structure upon entering the collection tube as shown in FIG. 1(b). In the microfluidic device, highly uniform A/O/W compound bubbles with a very low polydispersity (δ<3%) were generated at a typical frequency of several thousand hertz. PVA in the outer aqueous phase adsorbed to the oil-water interface and prevented the coalescence of the generated compound bubbles.

For a given device, the dimensions of the compound bubbles strongly depend on the flow rates of the middle (Qₘ) and outer phases (Qₒ) as well as the pressure of inner phase (Pᵢ), as shown in FIG. 1(c). An increase in the flow rate of the middle oil phase (Qₘ) generally leads to an increase in the thickness of the oil layer with no significant changes in the encapsulated bubble size. A drastic increase, however, leads to encapsulation of multiple gas bubbles that eventually coalesce, forming polydisperse compound bubbles. Changes in the flow rate of the continuous phase (Qₒ) and the pressure of the inner phase (Pᵢ) changed the size of the encapsulated bubbles, but they did not significantly influence the thickness of the oil shell as shown in FIG. 1(c). By tuning the flow rates of the three phases independently, the dimensions of A/O/W compound bubbles were controlled, thereby providing a method to tune the dimensions of nanoparticle-shelled bubbles.

To generate highly uniform bubbles encapsulated by a nanoparticle shell, it is critical to make and retain monodisperse and stable compound bubbles with a gas-core and an oil-shell suspended in water as a template. Previous studies, for example, have reported that the dewetting instability of the oil layer in water-in-oil-in-water (W/O/W) double emulsions prevented the generation of polymersomes, vesicles of amphiphilic diblock copolymers. To generate thermodynamically stable core-shell structures from three fluids, the spreading coefficient, defined as Sᵢ=γⱼₖ−γᵢⱼ−γᵢₖ≠j≠k=op, ip, mp (where γⱼₖ, γᵢⱼ, and γᵢₖ are the surface and interfacial tensions between j-k, i-j, and i-k fluids), of three phases must satisfy the following relationship: Sₒₚ<0, Sᵢₚ<0, and Sₘₚ>0 (the subscript, ip, and indicate the outer, inner, and middle phases, respectively). In our system, the spreading coefficients, determined using pendant drop tensiometry, were Sₒₚ=−27.1<0 mN/m, Sᵢₚ=−66.4<0 mN/m, and Sₘₚ=18.7>0 mN/m, satisfying conditions for the engulfment of one dispersed phase in the second phase. Due to the favorable wetting properties at each interface, the initial template configuration remained stable until the complete evaporation of the solvent.

### Example 2

### Characterization of Nanoparticle-Shelled Bubbles

The generation of compound bubbles in a device was monitored with a 10× objective using an inverted optical microscope (Nikon Diaphot 300) equipped with a high-speed camera (Phantom V7.1). The size distribution of bubbles upon collection was analyzed using an upright microscope (Carl Zeiss Axio Plan II) with a CCD camera (Qimaging Retiga 2000R Fast 1394). The average bubble size (Dₐᵥₑ) and polydispersity index (δ) were determined by measuring the sizes of at least 150 bubbles using the ImageJ software. The scanning electron microscope (SEM) images were taken using a Quanta 600 FEG Mark II. To prevent the accumulation of electric charge on the samples, samples were coated with a thin layer of gold. Fluorescent images were taken using a Nikon Diaphot 300 inverted microscope with a CCD camera and 75-watt Xenon lamp (Nikon).

Surface (air-liquid) and interfacial (liquid-liquid) tension measurements were performed using the pendent drop technique on a Ramé-Hart model 200 goniometer with DROPimage Advanced software to determine the spreading coefficients. Surface/interfacial tensions were measured from the shape of a droplet created at the flat tip of a stainless steel needle using a micrometer syringe (GS-1200, Gilmont Instruments). The reproducibility of tension measurements was within ±0.5 mN/m.

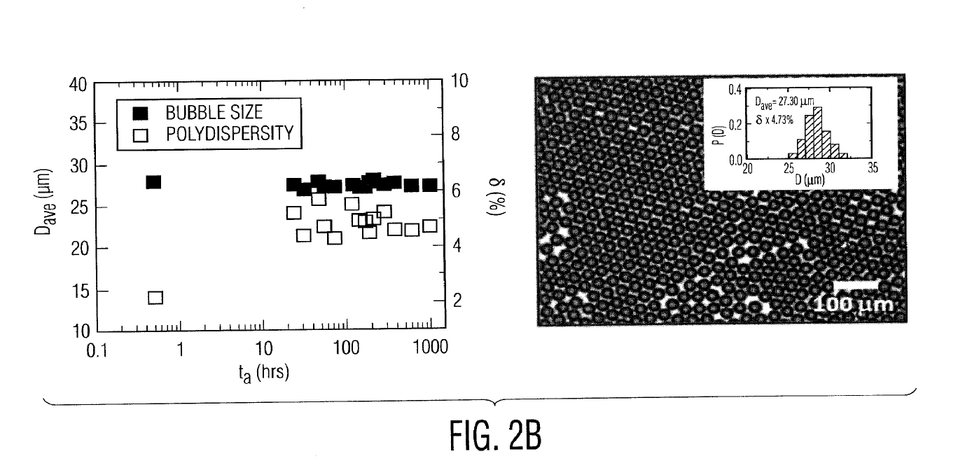

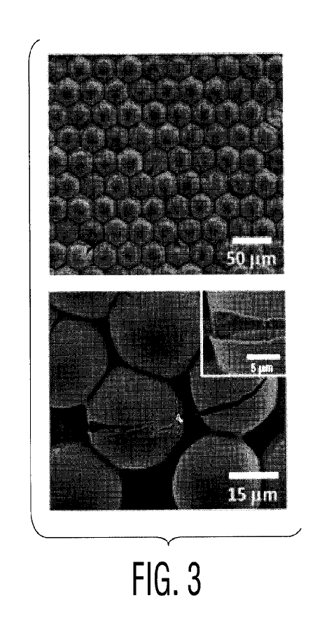

Nanoparticle-shelled bubbles generated upon the evaporation of the solvent, as shown in FIG. 2(a), were extremely stable against dissolution and coarsening. FIG. 2(b) displays the long-term stability of bubbles after collection from the microfluidic device; the average diameter (Dₐᵥₑ) and polydispersity (δ) of bubbles produced at Qₒ=120,000 μl/hr, Qₘ=1,000 μl/hr, and Pᵢ=82.7 kPa were plotted as a function of storage time tₐ. The initial diameter of compound bubbles, generated at the orifice of collection tube, was 36.2 μm (δ=1.2%). The diameter of compound bubbles decreased to 28.0 μm one hour after collection and did not undergo further change. Also, the polydispersity of bubbles increased from 1.2% to approximately 5.2% in the first 24 hours after collection and did not change significantly thereafter. The decrease in diameter and the increase in polydispersity in the early stages after collection likely resulted from the dissolution of gas into the continuous phase and the evaporation of oil. The formation of stiff nanoparticle shells, as evidenced by the presence of intact spherical hollow shells in the scanning electron microscopy (SEM) images in FIG. 3, arrested the shrinkage of bubbles, leading to the long-term stability for several months. The average diameter of hollow capsules as determined using SEM was 29.0 μm (δ=4.5%), consistent with the diameter of nanoparticle-shelled bubbles in solution (˜28 μm), as seen in FIG. 2(b). This observation indicates that the compound bubbles decrease to a terminal size upon removal of the solvent and that this event is accompanied by the formation of a stiff shell of randomly packed colloidal particles, providing excellent stability against dissolution and coarsening.

### Example 3

### Generation of Multicomponent Shelled Bubbles

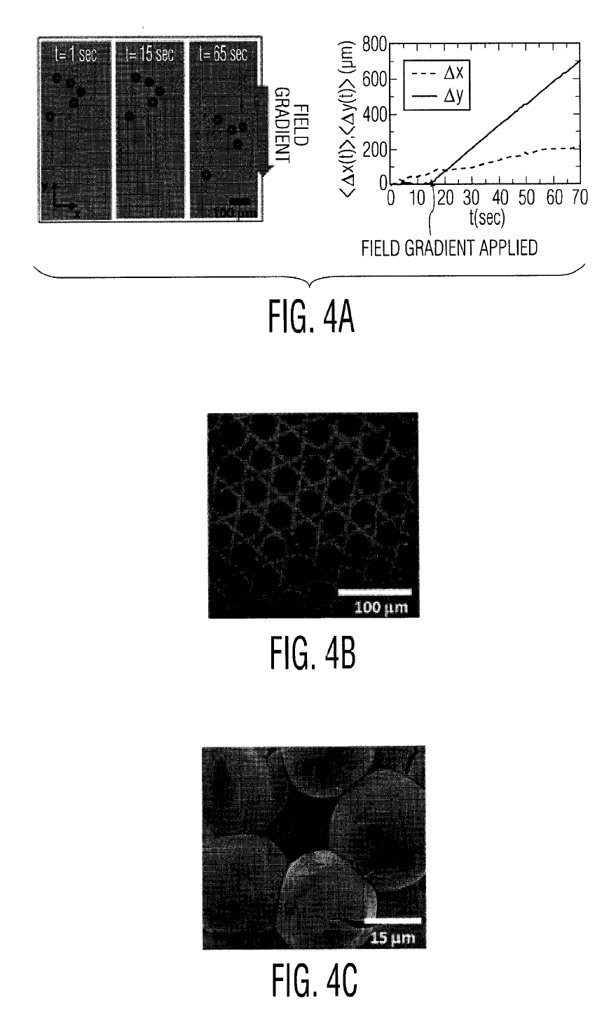

The unique feature of our approach is in the possibility of generating multi-component bubbles by incorporating a variety of materials into the silica nanoparticle shell. This can simply be achieved by suspending or dissolving functional materials into the oil layer containing silica nanoparticles and subsequently removing the solvent. A magnetically responsive bubble was generated, for example, by adding magnetic nanoparticles in the oil layer, as shown in FIG. 4(a). These magnetic bubbles could be readily manipulated using a magnetic field. In the absence of a magnetic field, the magnetic bubbles drifted in the x-direction, likely due to the curved water surface. Upon application of a magnetic field in the direction perpendicular to the spontaneous drift, the bubbles moved at a velocity of 12.8 μm/s in the direction of the field gradient. In addition to magnetic nanoparticles, a hydrophobic fluorescent dye, Nile Red, and a free fatty acid, palmitic acid, could be incorporated into the silica nanoparticle shells, as shown in FIGS. 4(b) and (c), respectively. These molecules coated the silica nanoparticles that formed the shell surrounding the gas bubbles after the removal of the solvent.

### Example 4

### Generation of Polymer-Shelled Bubbles

The A/O/W compound bubbles were generated using a glass-capillary microfluidic device that combines a co-flow and flow-focusing geometry. Briefly, two circular capillary tubes with inner and outer diameters of 0.58 mm and 1.0 mm (World Precision Instrument Inc.) were tapered to desired diameters using a micropipette puller (P-1000, Sutter Instrument Inc.) and a microforge (Narishige MF-830). The inner diameters of tapered tubes for the injection of a gas phase and the collection of bubbles were 2-8 μm and 80-150 μm, respectively. The outside of the glass capillary tube for inner fluid was hydrophobically functionalized with octadecyltrichloro-silane (OTS). This chemical treatment enhances the wettability of oil outside the capillary tube, and facilitates the formation of compound bubbles. The two tapered capillaries were inserted into a square capillary with an inner dimension of 1.0 mm, and subsequently sealed with epoxy.

For the fabrication of compound bubbles, one gas and two liquids were introduced into a microfluidic device using flexible Tygon tubing with a pressure regulator (ControlAir Inc.) and two syringe pumps (PHD, Harvard Apparatus), respectively. The inner gas phase was nitrogen (N₂), compressed air, carbon dioxide (CO₂), or helium (He), and the middle oil phase consisted of 1˜5 wt % PLGA polymer (75:25 Poly(DL-lactic-co-glycolic)acid, Ester Terminated, Durect Corp.) in dichloromethane. The outer water phase was composed of a mixture of 0˜50 vol % glycerol in 2 wt % PVA aqueous solution (PVA, 87-89% hydrolyzed, average M_{w}=13,000-23,000, Aldrich). The increase in viscosity using glycerol enabled the generation of compound bubbles with relatively low flow-rate of outer phase. The compound-bubble generation frequency (f_{cb}) was measured for calculating bubble dimensions such as shelf thickness and bubble radius. Generated compound bubbles flowed into the collection tube, and then one or two drops containing compound bubbles were collected into a large pool of water on a glass slide. This effectively lowers the concentration of PVA and glycerol in the continuous phase. The collected bubbles formed a monolayer at the top of water surface on the glass slide. The organic solvent was removed simply via evaporation, such as by heating the entire system. Because the bubbles floated to the water surface, the evaporation of the solvent occurred very rapidly. Based on the evaporation rate of the solvent, the solvent was expected to be removed in approximately 30 seconds. For the generation of PS- and PMMA-shelled bubbles, the middle phases were prepared by dissolving 2-4 wt % PS (M_{w}=400,000, M_{w}/Mₙ≦1.06, Pressure Chemical Co.) and 2-6 wt % PMMA (M_{w}=75,000, Scientific Polymer Products Inc.) in toluene, respectively.

The generation of compound bubbles in a device was monitored with a 10× objective using an inverted light microscope (Nikon Diaphot 300) equipped with a high-speed camera (Phantom V7.1) capable of 13,000 frames per second at the frame resolution of 800×200 pixels. The polymer-shelled bubbles formed from compound bubbles were imaged using an upright microscope (Carl Zeiss Axio Plan II) with a CCD camera (Qimaging Retiga 2000R Fast 1394). The percentage of deformed bubbles was determined by measuring the eccentricity of at least 250 bubbles using the ImageJ software and counting the number of particles below the eccentricity-threshold of 0.85. The scanning electron microscope (SEM) images were taken using a Quanta 600 FEG Mark II at an acceleration voltage of 5 kV.

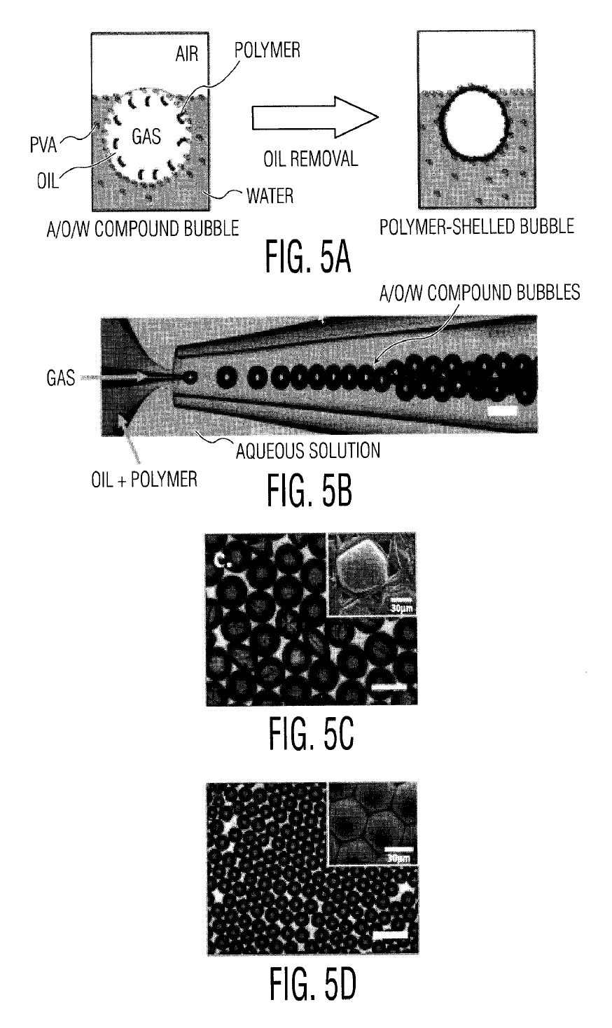

Polymer-shelled bubbles using a glassy biocompatible polymer, poly(DL-lactic-co-glycolic)acid (PLGA), were created using an A/O/W compound bubble as a template, as shown in FIG. 5(a). The monodisperse A/O/W compound bubbles were generated using a glass capillary microfluidic device (FIG. 5(b)). The inner phase (A) was nitrogen, and the outer phase (W) was a mixture of glycerol and 2 wt % poly(vinyl alcohol) (PVA) aqueous solution. The middle phase (O) comprised a volatile organic solvent, dichloromethane, in which PLGA was dissolved. Inner and middle fluid streams were hydrodynamically focused by the outer fluid, leading to the formation of compound bubbles with a polydispersity of less than 7%. Polymer-shelled bubbles were obtained by removing the solvent via evaporation. Accordingly, the bubble radius (R) and the thickness of bubble shell (h) could be precisely controlled.

Upon solvent removal, some polymer-shelled bubbles underwent deformation, exhibiting buckling or irreversible change in the shape of shells as seen in FIG. 5(c). Other bubbles with different dimensions, however, showed little deformation as shown in FIG. 5(d).

### Example 5

### Stability of Polymer-Shelled Bubbles

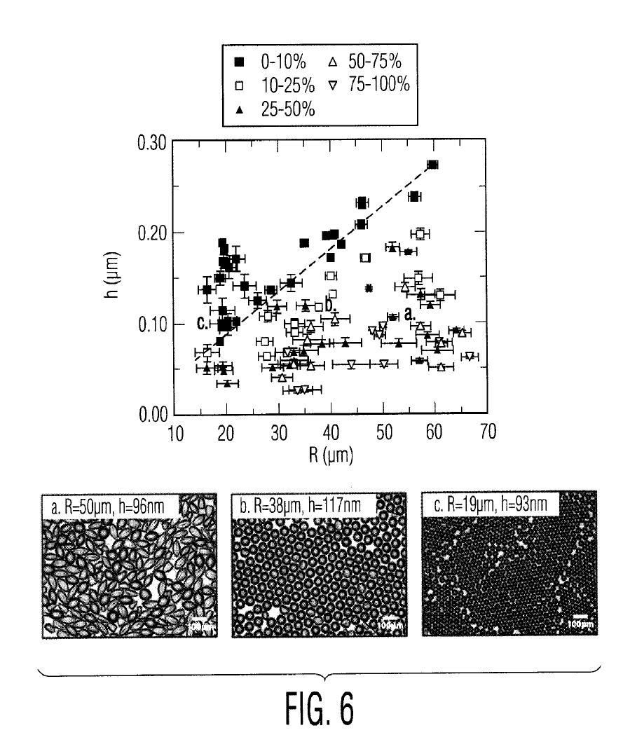

To characterize the effect of bubble dimension on the bubble deformation, the percentage of deformed bubbles as functions of shell thickness (h) and bubble radius (R) was illustrated in a state diagram (FIG. 6). For a given bubble radius, an increase in shell thickness led to an increase in the fraction of un-deformed bubbles, whereas for a given shell thickness, an increase in bubble size led to a decrease in bubble stability as shown in FIG. 6. This observation clearly indicates that the deformation of bubble shell is strongly influenced by the combined effect of the shell thickness and bubble radius.

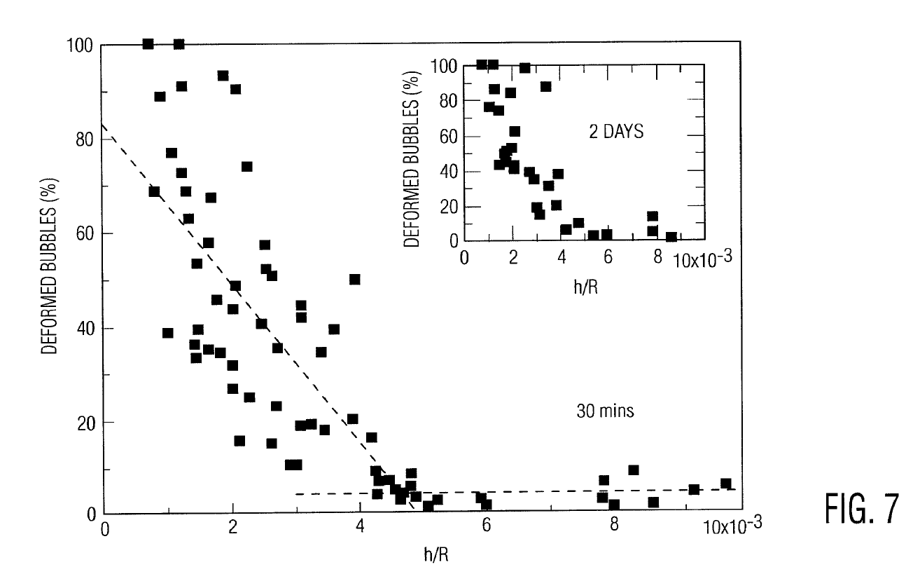

The stability of bubble shell was seen to correlate strongly with the ratio of shell thickness to bubble radius, h/R, as shown in FIG. 7. As h/R increased, the percentage of deformed bubbles decreased, reflecting an increase in the bubble stability against deformation. The fraction of un-deformed bubble plateaus as h/R was increased above a critical value. Consequently, the critical ratio of bubble shell thickness to bubble radius, (h/R)_{c}, for the onset of bubble shell deformation was determined to be 0.0046. The stable-unstable transition of bubble shell could be clearly delineated by a line with a slope of (h/R)_{c} as seen in FIG. 6 (this line going through the origin of the plot). There was no significant change in the fraction of deformed bubbles two days after preparation as shown in FIG. 7. The onset of bubble instability occurred within 30 minutes after preparation.

The deformation of bubble shell is due to the partitioning of gas (i.e., nitrogen) into the surrounding aqueous phase and the air above the aqueous phase by its diffusion through the elastic polymer shell that has formed after the solvent removal. The diffusion of gas through the elastic polymer shell leads to a pressure difference. The pressure difference, thus, will depend on the difference in the chemical potential of the gaseous species across the bubble shell but not strongly on the bubble dimension and the shell material. The pressure difference that induces shell deformation can be expressed using the following equation (1) describing the elastic instability of a spherical shell:

$$Δ P = \frac{2 {E}_{f}}{\sqrt{3 ( 1 - {v}^{2} )}} {( \frac{h}{R} )}_{c}^{2} , \qquad ( 1 )$$

where, ν and E_{f} are the Poisson's ratio and Young's modulus of the bubble shell.

Young's modulus of the polymer film in water was measured via strain-induced elastic buckling instability for mechanical measurement (SIEBIMM) method. This method determines the Young's modulus of polymer thin films based on their buckling on polydimethylsiloxane (PDMS) elastomer substrate induced by a uniaxial compression. To prepare the samples for testing, polymer films were transferred from silicon wafers onto PDMS substrates, as described previously. Briefly, PDMS substrates (Sylgard 184, Dow corning) were prepared by curing the degassed pre-polymer and initiator in a 10:1 w/w ratio for 2 hours at 75° C. Thin films of polymers were spin-coated from toluene solutions for PS and PMMA and chloroform solutions for PLGA onto plasma-treated silicon wafers. Specifically, the PLGA was deposited onto polyacrylic acid (PAA, 35 wt % aqueous solution, M_{w}=100,000, Sigma-Aldrich)-coated silicon wafer because of the difficulty in directly transferring the relatively hydrophilic polymer film to the PDMS substrate in water. The PAA dissolved in water aided in the releasing of the polymer film readily from the silicon wafer.

The polymer films on the PDMS substrates were buckled under water by applying a compressive strain using a pair of tweezers under a Nikon Diaphot 300 inverted microscope. The polymer films were left in water for at least 30 minutes. The wavelength of the buckling patterns was measured using a fast Fourier transform (FFT) of the optical image. The thickness of polymer films on the OTS-coated silicon-wafer in water was measured using an alpha-SE spectroscopic ellipsometer (J. W. Woollam Co., Inc.) with a home-made liquid cell. The OTS-layer prevents the delamination of the polymer film from Si wafers under water. With the measured values of buckling wavelength λ and film thickness h_{film}, the Young's modulus of polymer film was calculated using this equation (2):

$${E}_{f} = \frac{3 {E}_{s} ( 1 - {v}_{f}^{2} )}{1 - {v}_{s}^{2}} {( \frac{λ}{2 π {h}_{film}} )}^{3} , \qquad ( 2 )$$

where subscripts f and s indicate the polymer film and PDMS substrate, respectively, as shown in Table 1. The Young's modulus of PDMS (Eₛ) was independently measured using a dynamic mechanical analyzer (DMA; Q800 TA Instruments). Poisson's ratios of 0.33 and 0.5 were used for polymer and PDMS, respectively.

To confirm that different filling gases lead to different values of ΔP, PLGA-shelled bubbles were created using different gases. The values of ΔP were determined to be a strong function of the identity of filling gas as summarized in Table 1. Young's moduli of polymer films immersed in water for 30 minutes were measured using a buckling-based metrology.

TABLE 1
The pressure difference across the polymer shell determined
by the critical h/R and Young's moduli E_{f}

| Shell | Gas | (h/R)_{c} | E_{f} (GPa) | ΔP(kPa) |
|---|---|---|---|---|
| PLGA | N₂ | 0.0046 | 1.69 ± 0.54 | 43.8 ± 14.1 |
| | Air | 0.0038 | | 29.9 ± 9.6 |
| | CO₂ | 0.0068 | | 95.8 ± 30.7 |
| | He | 0.0054 | | 60.4 ± 19.4 |
| PS | N₂ | 0.0035 | 3.06 ± 0.36 | 45.8 ± 5.5 |
| PMMA | N₂ | 0.0040 | 2.25 ± 0.30 | 44.0 ± 5.9 |

While the generation of bubbles using compressed air led to a smaller value of ΔP compared to N₂-filled bubbles, the bubbles generated using helium (He) and carbon dioxide (CO₂) exhibited higher values of ΔP compared to N₂-filled bubbles. The fact that the bubbles filled with He, which has a low solubility in water, had a higher value of ΔP than N₂-filled bubbles indicates that it is not just the solubility of gas in water but also its transfer into the air above water that determines the pressure difference. Because of the scarcity of He in air, the pressure difference for He-filled bubbles is greater than that of N₂-filled bubbles. Thus, the results indicate that the partitioning of filling gas in the air above water surface as well as the solubility of gas in the is surrounding water are critical in determining the pressure difference across the bubble shells.

The pressure difference (ΔP) that leads to the deformation of polymer-shelled bubbles can be determined by measuring the Young's modulus of the polymer (E_{f}) as well as the critical ratio of shell thickness to bubble radius, (h/R)_{c}. As indicated above, ΔP should not depend on the properties of the shell material but rather on the identity of filling gas. To confirm this hypothesis, we determine the pressure difference inducing the deformation of nitrogen-filled bubbles by generating bubbles with different shell materials (Table 1). Glassy polymers, poly(methyl methacrylate) (PMMA) and polystyrene (PS), were used to determine the pressure difference across the bubble shell. The critical ratio (h/R)_{c} for each polymer was determined using the method described above. The Young's modulus (E_{f}) of each polymer in water was determined using a strain-induced elastic buckling instability for mechanical measurement (SIEBIMM) method. The pressure differences (ΔP) for N₂-filled bubbles obtained using the three different polymers were in an excellent agreement with each other as summarized in Table 1. The consistency obtained from N₂-filled bubbles generated with the three polymers clearly indicates that ΔP depends on the identity of the filling gas rather than on the shell material and bubble size. The bubble shell may have a small amount of residual solvent, which would lead to the overestimation of E_{f} as well as ΔP.

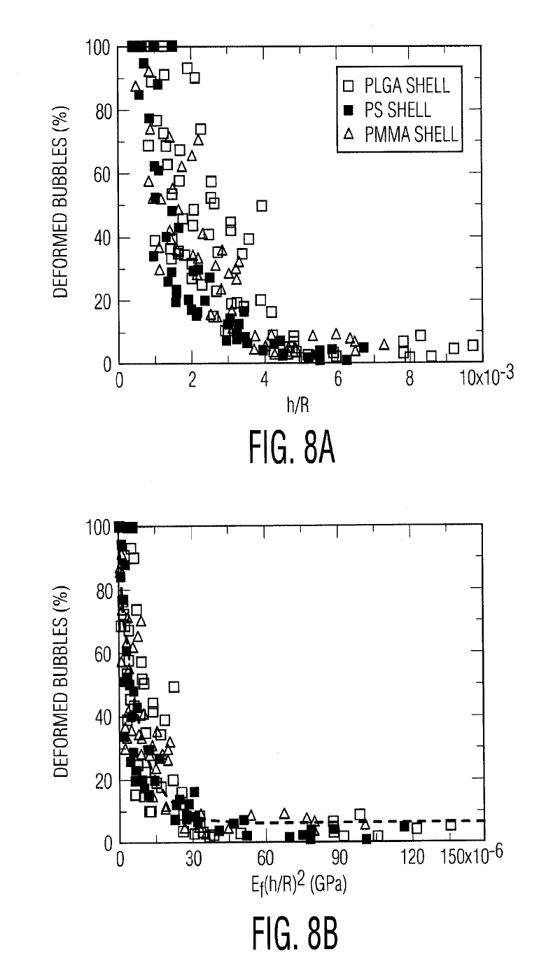

Equation 1 also suggests that a universal behavior may exist. It can be deduced that the deformation behavior of bubbles generated with different shell materials but with a given filling gas would scale as E_{f}(h/R)². When the fraction of deformed bubbles (N₂-filled) for each polymer (FIG. 8(a)) was scaled accordingly, the three curves superposed reasonably well onto a single master curve (FIG. 8(b)), indicating, indeed, the instability of polymer-shelled bubbles is a consequence of pressure difference induced by gas diffusion from the inside of the bubbles to the outside through the elastic polymer shell.

### Example 6

### Stability of Encapsulated Bubbles

Shelled bubbles were generated by introducing first an inner stream containing nitrogen (N₂), then a middle stream containing 3:2 TOL-ST and toluene, and lastly an outer stream containing 2 wt % PVA and 0.1M sucrose at flow rates of 16.4 psi, 1000 μl/hr and 100000 μl/hr, respectively, in accordance with the methods described in Examples 1 and 5. The shelled bubbles were subsequently stored in water at room temperature for over 360 days. No significant change in bubble size or dimension was observed while the bubbles were stored.

### Example 7

### Stability of Dried Shelled Bubbles Upon Re-Dispersion

Shell compound bubbles were generated by introducing first an inner stream containing nitrogen, then a middle stream containing toluene and silica nanoparticles, and lastly an outer stream containing PVA solution at flow rates of 5 psi, 2000 μl, and 35000 μl, respectively, in accordance with the methods described in Examples 1 and 5. The compound bubbles were collected in a Petri dish containing water, forming a monolayer. After solvent evaporation, the bubbles were rinsed with water, and encapsulated bubbles were formed. The encapsulated bubbles were subsequently dried and stored at room temperature for 2 days. Then, the dried encapsulated bubbles were re-dispersed in distilled water to disperse the bubbles. In this study, the stability of the dried encapsulated bubbles was evaluated.

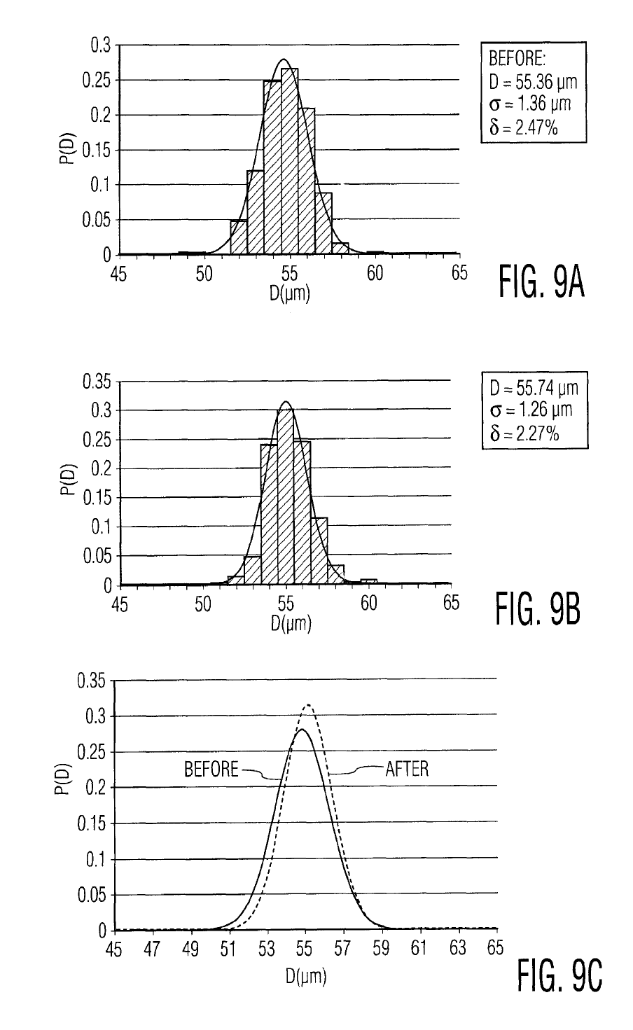

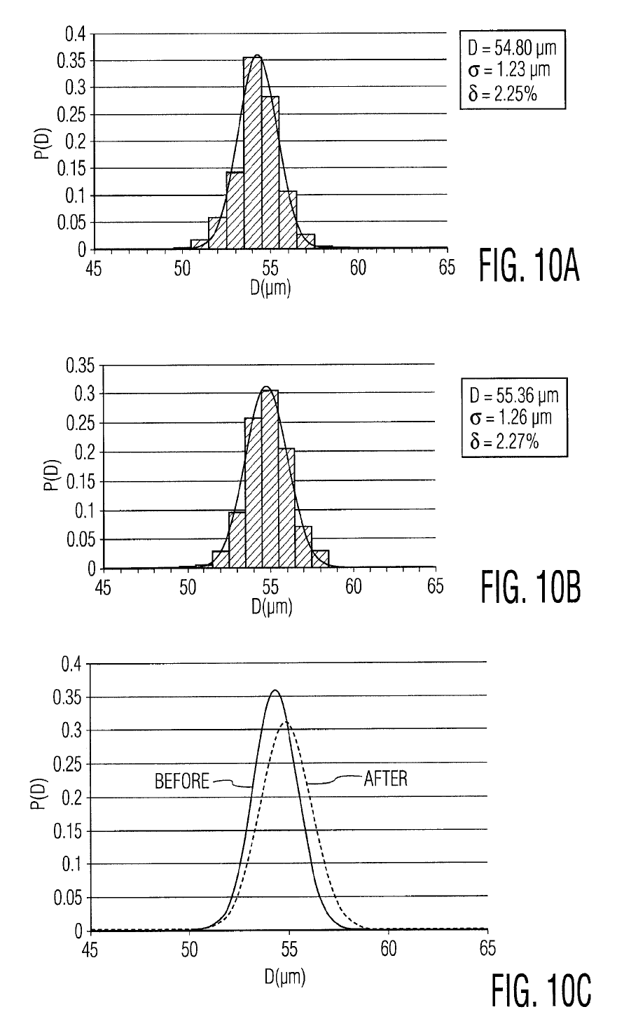

Images of two samples of dried encapsulated bubbles before drying and after re-dispersing were obtained, and the bubbles in the images were countered to generate probability size distribution for each sample. Size distribution graphs and curves are shown in FIGS. 9(A)-(C) for Sample 1 and in FIGS. 10(A)-(C) for Sample 2 before drying (FIGS. 9(A) and 10(A)) and after re-dispersing (FIGS. 9(B) and 10(B)). The size distribution curves before drying and after re-dispersing mainly overlapped taking into account of the measuring error (±1 pixel≈±100 nm) for Sample 1 (FIG. 9(C)) and for Sample 2 (FIG. 10(C)). There was no significant change in size distribution before drying and after re-dispersion.

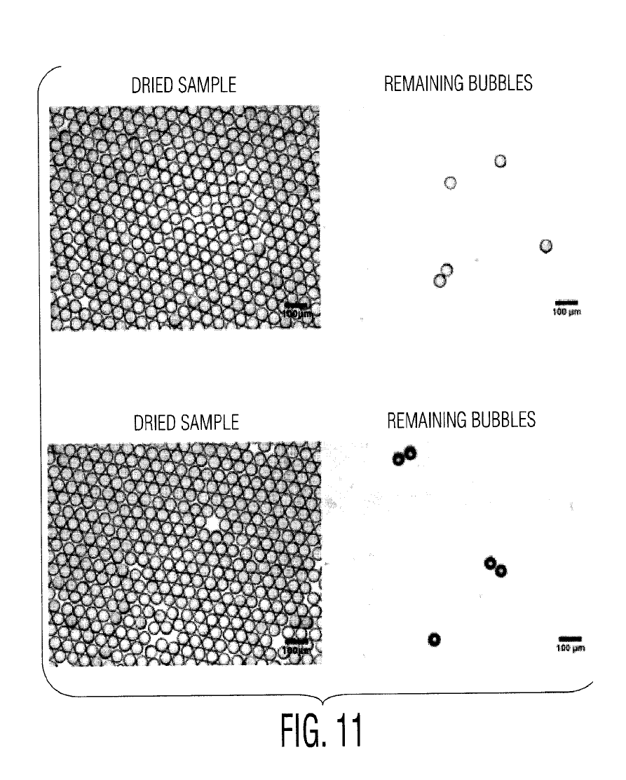

Images of the dried encapsulated bubbles (FIGS. 11(A) and 11(C)) and the remaining bubbles after re-dispersing (FIGS. 11(B) and 11(D)) are shown for Sample 1 (FIGS. 11(A) and 11(B)) and for Sample 2 (FIGS. 11(C) and 11(D)). The percentages of re-dispersed bubbles were about 98.68% and 98.67% for Samples 1 and 2, respectively.

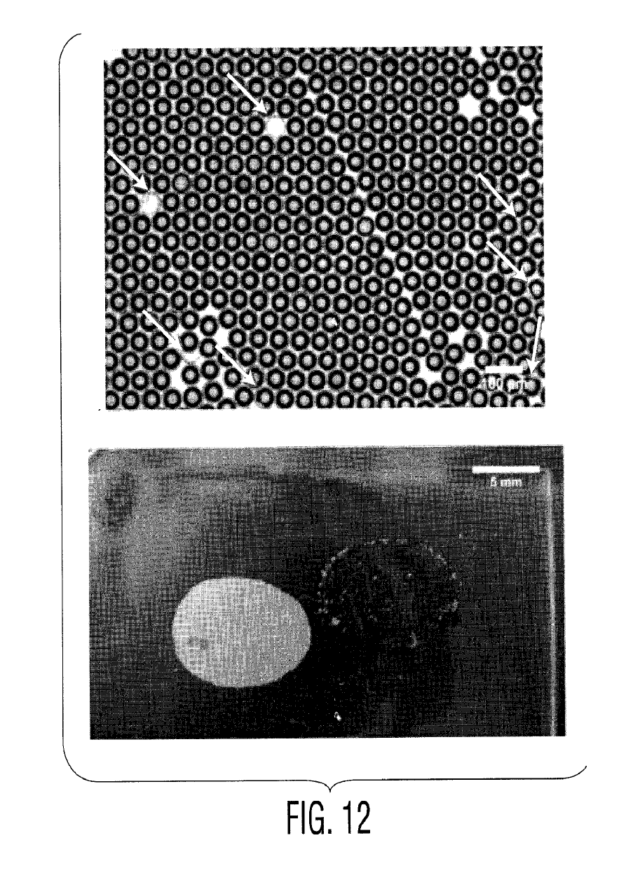

Percentages of stable bubbles after re-dispersing in Samples 1 and 2 were calculated from re-dispersed bubble pictures for Sample 1 and from total area covered by the bubbles for Sample 2. For Sample 1, about 97% of the re-dispersed bubbles were stable based on the image of the re-dispersed sample (FIG. 12(A), arrows is pointing to unstable bubbles). For Sample 2, the dried bubbles were re-dispersed on the same glass slide, which was initially covered by the dispersed bubbles. The area on the slide that was initially covered by the bubbles was marked. A picture was taken of the areas covered before and after re-dispersion (FIG. 12(B)). About 95% of the re-dispersed bubbles in Sample 2 were stable based on the total areas covered by the bubbles before and after re-dispersion (including non re-dispersed bubbles in the percentage).

Various terms relating to the systems, methods, and other aspects of the present invention are used throughout the specification and claims. Such terms are to be given their ordinary meaning in the art unless otherwise indicated.

The present invention is not limited to the embodiments described and exemplified above, but is capable of variation and modification within the scope and range of equivalents of the appended claims.

What is claimed:

1. A method of forming stable encapsulated bubbles, comprising:
(a) introducing an inner stream of a gas into a liquid-filled chamber from a circular exit orifice of a capillary tube;
(b) introducing, after step (a), a middle stream of a water immiscible liquid to the exit orifice within the chamber, wherein the water immiscible liquid comprises a shell forming material;
(c) introducing, after step (a), an outer stream of an aqueous liquid to the exit orifice within the chamber,
wherein the gas, the water immiscible liquid, and the aqueous liquid interact to form compound bubbles having an inner phase of the gas, a middle phase of the water immiscible liquid, and an outer phase of the aqueous liquid; and
(d) converting the middle phase of the compound bubbles into a shell, wherein the shell encapsulates the gas and is surrounded by the aqueous liquid, to form stable encapsulated bubbles.

2. The method of claim 1, wherein the diameter of the circular exit orifice is in the range of 2-5 μm.

3. The method of claim 1, wherein the gas is insoluble in the aqueous liquid.

4. The method of claim 1, wherein the encapsulated bubbles have a polydispersity of less than 3%.

5. The method of claim 1, further comprising modifying at least one of:
(a) the ratio of the shell thickness to the bubble radius;
(b) the stiffness of the bubble shell; and
(c) the identity of the gas;
to improve the stability of the encapsulated bubbles.

6. The method of claim 5, wherein the modifying step consists of modifying the ratio of the shell thickness to the bubble radius and modifying the ratio of the shell thickness to the bubble radius consists of adjusting the relative volumetric flow rates of at least one of the inner stream, the middle stream, and the outer stream.

7. The method of claim 1, wherein the water immiscible liquid further comprises an active pharmaceutical ingredient.

8. The method of claim 1, wherein the water immiscible liquid further comprises nanoparticles.

9. The method of claim 1, wherein the water immiscible liquid further comprises a solvent and the converting step comprises
(a) collecting the compound bubbles substantially in a monolayer; and
(b) removing the solvent from the middle phase of the compound bubbles in the monolayer to convert the middle phase into the shell.

10. The method of claim 1, further comprising storing the encapsulated bubbles for at least one year.

11. A method of forming stable dried encapsulated bubbles, comprising:
(a) introducing an inner stream of a gas into a liquid-filled chamber from a circular exit orifice of a capillary tube;
(b) introducing, after step (a), a middle stream of a water immiscible liquid to the exit orifice within the chamber, wherein the water immiscible liquid comprises a shell forming material;
(c) introducing, after step (a), an outer stream of an aqueous liquid to the exit orifice within the chamber,
wherein the gas, the water immiscible liquid, and the aqueous liquid interact to form compound bubbles having an inner phase of the gas, a middle phase of the water immiscible liquid, and an outer phase of the aqueous liquid;
(d) converting the middle phase of the compound bubbles into a shell, wherein the shell encapsulates the gas and is surrounded by the aqueous liquid, to form stable encapsulated bubbles; and
(e) removing the aqueous liquid from the stable encapsulated bubbles of step (d) to form stable dried encapsulated bubbles.

12. The method of claim 11, further comprising re-dispersing the dried encapsulated bubbles in an aqueous solution to form re-dispersed encapsulated bubbles.

13. The method of claim 12, wherein, upon re-dispersion, at least 80% of the dried encapsulated bubbles form stable re-dispersed encapsulated bubbles.

14. A composition comprising the encapsulated bubbles obtained by the method of claim 1.

15. A composition comprising the dried encapsulated bubbles obtained by the method of claim 11.

16. A composition comprising the re-dispersed encapsulated bubbles obtained by the method of claim 12.

17. A method of treating a subject, comprising administering to the subject a composition comprising the encapsulated bubbles obtained by the method of claim 7, wherein the active pharmaceutical ingredient is present in a therapeutically effective amount.

18. The method of claim 17, wherein the composition is administered by a route selected from the group consisting of oral, subcutaneous, intravenous, intranasal, transdermal, intraperitoneal, intramuscular, intrapulmonary, vaginal, rectal, and intraocular routes.

19. The method of claim 17 further comprising:
(a) delivering the encapsulated bubbles to a target location in the subject; and
(b) releasing the active pharmaceutical ingredient from the encapsulated bubbles at the target location.

20. A method of enhancing ultrasound imaging in a subject, comprising administering to the subject a composition comprising the encapsulated bubbles obtained by the method of claim 8.

21. The method of claim 1, wherein the middle stream is introduced in step (b) before the outer stream is introduced in step (c).

22. The method of claim 1, wherein the water immiscible liquid further comprises a functional material.
